(19) United States
(12) Patent Application Publication
(10) Pub. No.: US 2024/0201166 A1
(43) Pub. Date: Jun. 20, 2024

(54) DIAGNOSIS AND TREATMENT OF CONGESTIVE COLON FAILURE (CCF)

(71) Applicant: Vikas Khurana, South Abington Township, PA (US)

(72) Inventor: Vikas Khurana, South Abington Township, PA (US)

(21) Appl. No.: 18/286,364

(22) PCT Filed: Apr. 11, 2022
(86) PCT No.: PCT/US2022/024230

§ 371 Date: Oct. 10, 2023

Related U.S. Application Data
(60) Provisional application No. 63/201,076, filed on Apr. 11, 2021.

(51) Int. Cl. G01N 33/50; A61B 5/00; G16H 40/67; G16H 50/20

(52) U.S. Cl. CPC G01N 33/50; A61B 5/42; A61B 5/6891; G16H 40/67; G16H 50/20; G01N 2333/92; G01N 2333/926; G01N 2800/52

(57) ABSTRACT

A method of treatment for congestive colon failure (CCF) including evaluating an origin of a lipase and/or an amylase from a biological fluid sample from the subject, optionally analyzing a level of the lipase and/or an amylase from the biological fluid sample from the subject, optionally imaging a colon and a pancreas of the subject and providing laxatives, an anti-fungal composition, fat, a composition selected from a group comprising glucosamine, methylsulfonylmethane (MSM) rhamnan sulfate, Sulodexide, Rosuvastatin, Metformin, Hydrocortisone, Antithrombin, Etanercept, Heparin, Albumin, Fresh frozen plasma (FFP), Poloxamer-188, vitamins, trace elements, a locally acting vasodilator, anaerobic microbial culture, pickled fruits, pickled vegetables, preserved meat, and fermented juices or a combination thereof based upon results of the first three steps.

### FIELD OF INVENTION

The present disclosure relates to compositions and methods for diagnosing and treating colon disorders. The present disclosure specifically relates to methods and compositions for correctly diagnosing congestive colon failure and differentiating it from other gastrointestinal disorders with similar or overlapping symptoms.

### BACKGROUND

Pancreatitis occurs as a consequence of premature, intra-pancreatic activation of pancreatic proenzymes. These include chymotrypsinogen, procolipase, prophospholipase A2, and proelastase. These proenzymes that are synthesised by the acinar cells and stored in vesicles are known as zymogens. They are released into the pancreatic duct and activated at the brush border of the duodenal enterocytes.

Acute pancreatitis can be a diagnostic challenge given the non-specific nature of the symptoms and widely varying results of investigations. The diagnosis typically involves a combination of history and examination, abnormal laboratory investigations and radiological evidence of pancreatic inflammation.

The diagnosis of acute pancreatitis usually requires a combination of clinical, laboratory and radiological findings. A number of international guidelines have suggested that two of the following three features are required for the diagnosis: abdominal pain consistent with acute pancreatitis (acute onset of persistent severe epigastric pain often radiating to the back), serum lipase activity (or amylase activity) at least three times greater than the upper limit of normal, and characteristic findings of acute pancreatitis on abdominal ultrasound (a CT scan or MRI is considered if the diagnosis is uncertain).

Serum amylase in humans is secreted as two isoforms: one by the salivary glands (s-amylase) and the second by the pancreas (p-isoamylase). Its predominant action is to digest starch, glycogen, and other related polysaccharides. Almost all laboratories currently measure total serum amylase, and the assay is a chemical reaction that cannot distinguish between the different isoenzymes; therefore, the result includes a combined level of all isoenzymes. The reference range is typically 20-300 U/L, but it varies with age and gender. It also varies between the different laboratories where the test is done, despite attempts to adopt standardized reference methods. Most guidelines now suggest that an amylase concentration of 2-4 times the upper limit of normal is optimal for diagnostic accuracy, but this may reduce the sensitivity of the test to as low as 60%. In acute pancreatitis, amylase can rise rapidly within 3-6 hours of the onset of symptoms and may remain elevated for up to five days. However, it has a short half-life of 12 hours so the concentration can normalize within 24 hours. This significantly reduces its value as a diagnostic test relatively early in the clinical course. The same holds true for lipase activity measurement, as the assays being done by clinicians do not distinguish between the different isozymes for lipase.

Many conditions can increase serum amylase as it is not specific for pancreatitis. These conditions include various intra- and extra-abdominal illnesses and drugs. Macroamylasaemia is an uncommon condition in which amylase rises because its clearance is reduced. Since up to 60% of the total serum amylase originates from non-pancreatic sources, the diagnostic accuracy for acute pancreatitis may increase by measuring the pancreatic isoenzyme. However, since the pancreatic isoenzyme levels also increases in many of the other nonpancreatic causes of hyperamylasaemia, there are not many studies showing the benefits of measuring the pancreatic isozyme specifically. Hence, total amylase, along with lipase, is still considered the hallmark for diagnosing acute pancreatitis. Human Serum amylase is secreted in specific isoforms by the salivary glands (s-amylase) and pancreas (p-isoamylase).

Serum lipase has now replaced amylase as the biochemical test of choice in acute pancreatitis. With an important role in fat digestion, the tissue concentration of lipase in the pancreas is 100-fold higher than in other tissues such as the duodenum, stomach, adipose tissue, and lung. Serum lipase typically increases 3-6 hours after the onset of acute pancreatitis and usually peaks at 24 hours. Unlike amylase, there is significant reabsorption of lipase in the renal tubules so the serum concentrations can remain elevated for up to 8-14 days. This means it is a far more useful diagnostic marker than amylase when the clinical presentation or testing has been delayed by more than 24 hours. Serum lipase also has a greater sensitivity than amylase in patients with alcoholic pancreatitis. Several studies suggest its sensitivity is 85-100%. There are several other conditions that can elevate lipase including other pancreatic diseases, cholecystitis, intestinal ischemia, renal impairment, and malignancy. However, the test's specificity has been shown to be higher than amylase testing in several studies. Depending on the defining criteria and value cut-offs, specificity of lipase may be higher than 95%. Like serum amylase, there is some variability in the reference ranges for lipase, and debate about the optimal cut-off value that should be used to diagnose acute pancreatitis. Most guidelines now recommend 2-3 times the upper limit of normal as the most appropriate cut-off.

Over the last decade there has been a significant increase in Chemical Pancreatitis in patients presenting with acute abdominal pain, a condition where lipase elevation and amylase elevation is noted on laboratory evaluation, but CT scan does not show any evidence of pancreatic inflammation. This has raised a significant concern that the source of increase in levels of lipase and/or amylase may not be the pancreas but might be some other source.

After extensive evaluation with CT scans looking at multiple research papers and clinical review of the history it was noticed that many of these patients had significant retention of stool in the colon. Although they had stool retention in the colon, they were presenting with diarrhea alternating with constipation. Upon investigation it was found that many of these patients had significant stool retention in the right side of the colon. In addition, while colonic perforation or colitis was reported, importance of segmental stool retention and their correlation with symptom complex has not been studied, reported, or recognized thus far. Moreover, the stool on the left side of the colon may be absent if a patient had recent bowel movement, thus further complicating the diagnosis. This creates a significant diagnostic challenge and most of these patients never get their stool retention addressed. This consortium of symptoms, associated with long-term stool retention caused from the inability of normal colonic peristalsis, has been termed as “congestive colon failure (CCF)”.

It was also noted that aggressive treatment of stool retention with laxatives caused the resolution of their symptoms and eventually resulted in the correction of serum lipase and amylase elevation. This is partly or completely due to resolution of the suspected fungal infection accompanying CCF, which was probably the original cause of elevation of lipase and/or amylase in the patient's body.

Investigation of the mechanism of lipase elevation and amylase elevation in CCF patients can be connected to the symptoms and it was found that the current methodology for testing amylase and lipase was to check for their enzymatic reaction without testing for the specificity of the enzyme coming from the human or foreign source.

Moreover, it was found that increased pathogenicity of fungus was correlated with more aggressive production of lipase enzyme.

It is well known that there is small bowel fungal overgrowth (SBFO) in some individuals. But the causes, diagnosis, and treatment of this has not made much progress. It is also not known if fungal overgrowth is in the small bowel or the colon.

Therefore, there is a need to for accurate diagnostic test and treatment for CCF.

### SUMMARY

In an aspect of the present disclosure, a method of treatment for congestive colon failure (CCF) in a subject provided.

The method involves evaluating an origin of a lipase and/or an amylase from a biological fluid sample from the subject, optionally analyzing a level of the lipase and/or an amylase from the biological fluid sample from the subject, optionally imaging a colon and a pancreas of the subject and providing laxatives, an anti-fungal composition, fat, a composition selected from a group comprising glucosamine, methylsulfonylmethane (MSM) rhamnan sulfate, Sulodexide, Rosuvastatin, Metformin, Hydrocortisone, Antithrombin, Etanercept, Heparin, Albumin, Fresh frozen plasma (FFP), Poloxamer-188, vitamins, trace elements, a locally acting vasodilator, anaerobic microbial culture, pickled fruits, pickled vegetables, preserved meat, and fermented juices or a combination thereof based upon results of the first three steps.

In another aspect of the method to treat congestive colon failure of the present disclosure, origin of the lipase and/or amylase is evaluated by the way of analyzing biomarkers related to lipase and/or amylase.

In another aspect of the method to treat congestive colon failure of the present disclosure, imaging is done by the way of magnetic resonance imaging (MRI), X-Ray, CT-scan, ultrasound, thermal imaging, nuclear imaging, physical exam, or a combination thereof.

In another aspect of the method to treat congestive colon failure of the present disclosure, the fat is selected from a group comprising non-absorbable fats, wax esters, and fat from butter fish, or a combination thereof.

In another aspect of the method to treat congestive colon failure of the present disclosure, further involves alternating between fiber and fiber free diet wherein fiber diet comprises of dietary and supplemental fiber.

In an aspect of the present disclosure, a kit to diagnose congestive colon failure (CCF) is provided.

The kit includes at least a first reagent to identify a fungal species, at least a second reagent to identify fungal enzymes and a test strip made of a membrane, adhesively attached to a solid base with the first and second reagents immobilized on it.

In an aspect of the present disclosure, a method to differentiate between congestive colon failure and pancreatitis in a subject is provided.

The method involves evaluating an origin of a lipase from a bodily fluid of the subject, imaging a pancreas and a colon and differentiating between pancreatitis and congestive colon failure based on initial two steps.

In an aspect of the present disclosure, a method to detect congestive colon failure in a subject is provided.

The method includes administering a set of sensors in the subject through the subject's mouth, tracking a movement of the set of sensors through a gastro-intestinal tract of the subject, mapping the movement of each sensor over time to compile a composite map of a movement of stool over time and detecting congestive colon failure based on the composite map.

In another aspect of the method to detect congestive colon failure of the present disclosure wherein the stool flow over time is tracked using a set of Radio Frequency Identification (RFID) sensors.

In another aspect of the method to detect congestive colon failure of the present disclosure wherein the Radio Frequency Identification sensors are hygroscopic and provide real-time information.

In an aspect of the present disclosure a toilet system is provided.

The toilet system includes an input unit configured to determine a set of attributes associated with a human waste and generate a first signal based on the set of attributes of the human waste. A processing circuitry coupled with the input unit and configured to receive the first signal from the input unit and analyze the first signal to determine an intestinal health of the subject. An output unit coupled with the processing circuitry and configured to relay the gastro-intestinal health of the subject on an audio-visual device of the output unit and interact with users to receive instructions from the user.

In another aspect of the toilet system of the present disclosure, the input unit consists of an imaging unit configured to capture images a set of sensors configured to sense temperature, texture, color, consistency, flow or a combination thereof; and an audio unit configured to receive sound.

In another aspect of the toilet system of the present disclosure, the set of attributes of the human waste include smell, consistency, texture, color, flow, temperature.

### BRIEF DESCRIPTION OF DRAWINGS

The drawing/s mentioned herein disclose exemplary embodiments of the claimed disclosure. Other objects, features, and advantages of the present disclosure will be apparent from the following description when read with reference to the accompanying drawing.

FIG. 1 illustrates a flowchart that depicts a method (100) of treatment for congestive colon failure (CCF) in a subject, according to an embodiment herein;

FIG. 2 illustrates a flowchart that depicts a method (200) to differentiate between congestive colon failure and pancreatitis in a subject, according to an embodiment herein;

FIG. 3 illustrates a flowchart that depicts a method (300) to detect congestive colon failure in a subject, according to an embodiment herein;

FIG. 4 illustrates a system, according to an embodiment herein;

FIGS. 5A and 5B illustrates a clip device, according to an embodiment herein; and

FIG. 6 illustrates a flowchart that depicts a method of detecting Congestive Column Failure according to an embodiment herein.

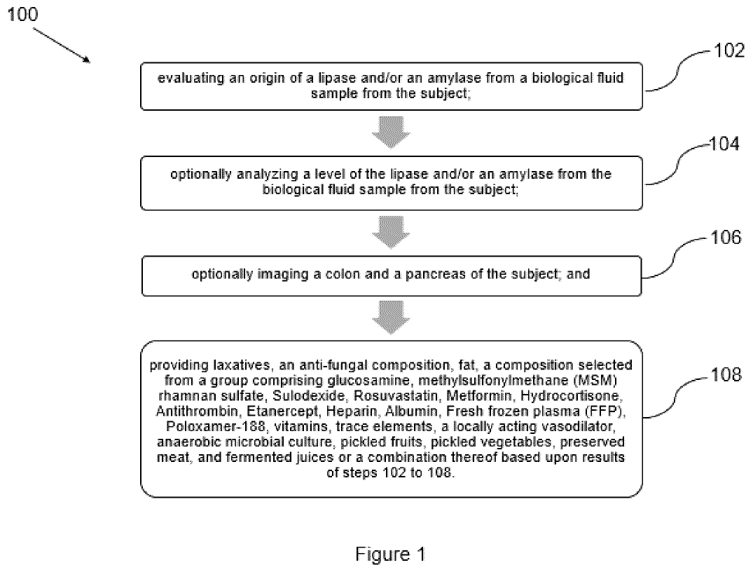

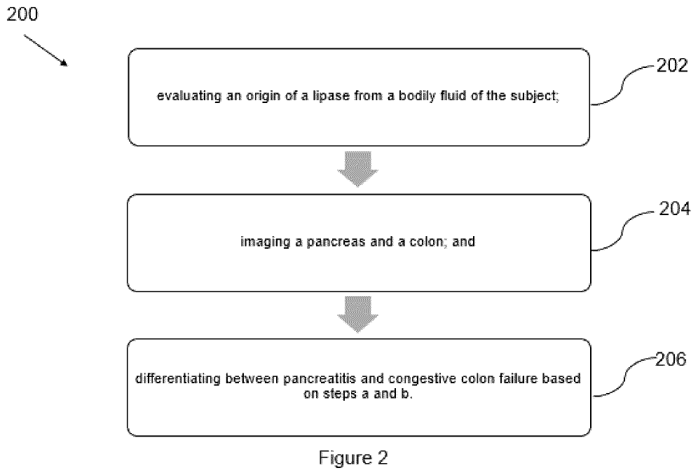

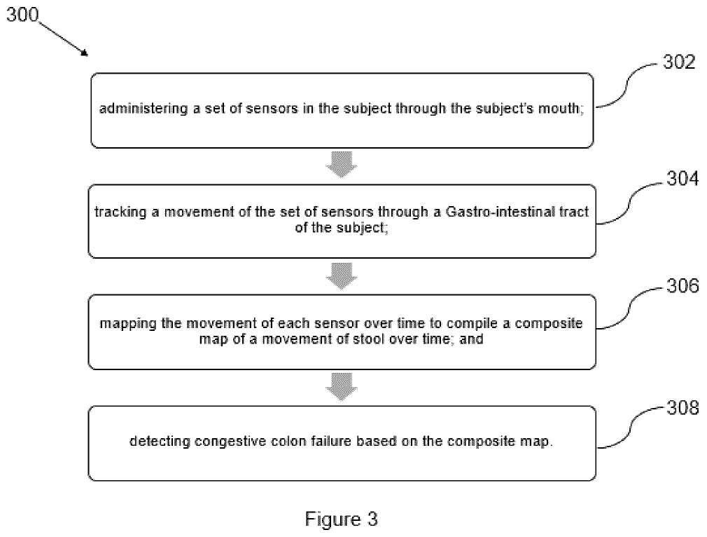

To facilitate understanding, like reference numerals have been used, where possible to designate like elements common to the figures.

### DETAILED DESCRIPTION

This section is intended to provide explanation and description of various possible embodiments of the present invention. The embodiments used herein, and the various features and advantageous details thereof are explained more fully with reference to non-limiting embodiments illustrated in the accompanying drawing/s and detailed in the following description. The examples used herein are intended only to facilitate understanding of ways in which the embodiments may be practiced and to enable the person skilled in the art to practice the embodiments used herein.

The terms “RFID chips” and “RFID sensors” are used interchangeably used across the context.

### Definitions

Congestive Colon Failure (CCF): Colonic muscle movement or peristalsis is impacted in the same way as the heart muscle when excessive dilation of the hollow organ takes place. To move anything in the colon, a fixed amount of muscular pressure must be generated to cause the stool to be pushed forward. As the stool starts collecting in the colon, the colon starts dilating which translates to the thinning of the bowel wall. This leads to a thinned-out muscle which is unable to generate pressure to move the stool in the colon. In other words, the pressure generated by the thinned-out muscle is incapable of moving the total amount of stool present in the colon relative to a normal muscle. Eventually the colonic muscle fails, as the maximum amount of pressure it can generate does not move the stool at all. This leads to colonic failure or “Congestive Colon Failure (CCF)”. This consortium of symptoms, associated with long-term stool retention, has been termed as “congestive colon failure”.

Pancreatitis: Pancreatitis is the inflammation of the pancreas. Acute pancreatitis (AP) is one of the most common gastrointestinal causes for hospitalization in the United States. In 2015, AP accounted for 390,940 hospitalizations making it one of the most frequent causes of gastrointestinal hospitalizations in the nation with the annual incidence only expected to increase over time. Despite recent advances in gastroenterology, AP continues to be associated with substantial mortality, morbidity, and healthcare resource utilization.

AP is the inflammation of the pancreas that is often associated with systemic inflammatory response syndrome (SIRS) that may further impair the function of other organs.

Gastroparesis: Gastroparesis is defined as “a chronic symptomatic disorder of the stomach characterized by delayed emptying without evidence of mechanical obstruction.” Thus, three elements are central to the clinical diagnosis of gastroparesis: related symptoms, gastric emptying delay, and the absence of another organic explanation for the patient's symptoms (for example, obstruction).

Lipases: Lipases catalyzes the hydrolysis of ester bonds of triacylglycerols, resulting in the release of fatty acids. In almost all organisms, lipases play essential roles in lipid metabolism, including digestion, transport, and the processing of dietary lipids

Lipolytic enzymes have also been implicated in the virulence of fungal pathogens; the contribution of lipases in fungal pathogenesis has been extensively characterized in Candida species. C. albicans possesses at least 10 lipase-encoding genes, the expression of which is largely influenced by the stage of infection. In C. parapsilosis, lipases are responsible for the destruction of epidermal and epithelial tissues. Lipases also play important roles in the virulence of skin-associated lipophilic fungal pathogens of the Malassezia spp. The gene that encodes for lipases in M. furfur, M. pachydermatis, and M. globosa has been identified, and recent genome sequencing efforts have revealed at least 14 lipase-encoding genes in M. globosa.

The term “isoenzymes” or “isozymes” refers to enzymes that catalyse the same reaction and occur in the same species but have different physio-chemical properties. Isoenzymes are distinct, and often readily separable forms of an enzyme expressed by the same organism. Isozymes catalyse the same chemical reaction, but typically differ with respect to their primary structure, intracellular location, and physiological role. Multimeric isozymes may be the products of different genes or differential gene-splicing events.

Rub and denudation of colonic barrier defenses is herein defined as decline or loss of mucosal defenses in the colon, because of lack of fat, damaging chemicals including whitening agents or bleaching agents used in food. The colonic mucosa is not provided with an adequate amount of free fatty acids and other nutrients, which are required for the growth and health of the colonic mucosa mucosa and glycocalyx present on the cell lining. Damage to glycocalyx permits infiltration of blood circulating foreign bodies that can cause gastrointestinal diseases.

### Embodiments

The present disclosure relates to methods and compositions for determining and treating colon congestive failure (CCF) in a subject.

In an embodiment of the present disclosure, a method (100) of treatment for congestive colon failure (CCF) in a subject is provided.

The method involves evaluating (102) an origin of a lipase and/or an amylase from a biological fluid sample from the subject, optionally analyzing (104) a level of the lipase and/or an amylase from the biological fluid sample from the subject, optionally imaging (106) a colon and a pancreas of the subject and providing (108) laxatives, an anti-fungal composition, fatty acids, fats, a composition selected from a group comprising glucosamine, methylsulfonylmethane (MSM) rhamnan sulfate, Sulodexide, Rosuvastatin, Metformin, Hydrocortisone, Antithrombin, Etanercept, Heparin, Albumin, Fresh frozen plasma (FFP), Poloxamer-188, vitamins, trace elements, a locally acting vasodilator, anaerobic microbial culture, pickled fruits, pickled vegetables, preserved meat, and fermented juices or a combination thereof based upon results of the first three steps.

In another embodiment of the method (100) to treat congestive colon failure of the present disclosure, origin of the lipase and/or amylase is evaluated by the way of analyzing biomarkers related to lipase and/or amylase.

In another embodiment of the method (100) serum lipase and/or amylase for non-pancreatic origin in a subject is analyzed, wherein the subject exhibits elevated levels of serum lipase and/or amylase compared to a control.

In another embodiment, the non-pancreatic serum lipase and/or amylase in the method described herein is of fungal origin.

In another embodiment, the non-pancreatic serum amylase in the method described herein is of fungal origin.

In yet another embodiment, the non-pancreatic serum lipase in the method described herein is of fungal origin.

In another embodiment, the fungal enzymes (amylase and/or lipase) enter the circulation in CCF patients.

In an embodiment of the present disclosure, a method to determine CCF in a subject is provided. The method comprising analyzing symptoms of the subject, testing a blood serum level of lipase, testing a blood serum level of amylase, imaging a pancreas and colon of the subject, culturing a stool sample of the subject.

In yet another embodiment, pain pattern of the subject is not diagnostic of pancreatitis.

In another embodiment, biochemical markers that parallel the elevation of pancreatic serum lipase and/or amylase is used for the detection of CCF. In another embodiment the biochemical markers are from an immune response caused by fungal overgrowth/invasion in the subject.

In another embodiment the biochemical marker excludes human origin. In yet another embodiment the biochemical marker is of human origin.

In yet another embodiment, analysis of the biochemical marker helps to determine fungal overgrowth in intestines.

In one embodiment, this is due to damage to the blood gut barriers by the fungal lipase and amylase because of the heavy load of the invading fungus. In one embodiment, the fungal cells/spores are not able to enter due to the natural immune defenses of the body.

In another embodiment, the fungal enzymes in CCF patients enters the bloodstream through colon microvasculature.

In another embodiment, the non-pancreatic serum lipase and/or amylase in the method described herein is of colonic origin.

In yet another embodiment, imaging in the method (100) is done by the way of magnetic resonance imaging (MRI), X-Ray, CT-scan, ultrasound, thermal imaging, nuclear imaging, and/or any other imaging modality, and/or physical exam.

In another embodiment, the method (100) further comprises doing at least one further test to confirm fungal infection of the colon.

In yet another embodiment, supranormal doses of laxatives are used for treating constipation comprising 17 grams of Miralax spread across 6-13 doses taken every 30 minutes.

In another embodiment the method (100), further comprises, administering a lipase inhibitor in the subject before administering fatty acids to allow delivery of fatty acids directly to the colon.

In another embodiment, a consortium of anaerobic culture is administered in the method (100).

In yet another embodiment the method (100), further comprises of alternating between fiber and fiber free diets.

In another embodiment, the subject has at least one symptom associated generally with acute pancreatitis.

In another embodiment, at least one symptom is severe abdominal pain.

In another embodiment, at least one symptom is severe acute abdominal pain.

In another embodiment, at least one symptom is chronic recurrent abdominal pain.

In another embodiment, at least one symptom is chronic abdominal pain.

In another embodiment, at least one symptom is severe acute abdominal pain superimposed on chronic abdominal pain.

In another embodiment, at least one symptom is elevated levels of serum amylase and/or lipase compared to a control.

In another embodiment, the subject exhibits elevated levels of serum amylase and/or lipase as well as severe abdominal pain.

In yet another embodiment, unmodified fat is delivered to the colon by the way of a smart pill, enteric coating, micelles, pH-based pill or a combination thereof.

In another embodiment, lipase activity is detectable in stool in patients with congestive colon failure.

In another embodiment, amylase activity is detectable in stool in patients with congestive colon failure.

In another embodiment, lipase or amylase precursors or partially broken-down protein is detectable in stool in patients with congestive colon failure.

In one embodiment, at least one further test to confirm fungal infection of the colon is done by taking a biopsy of the colon to detect fungal infection of the colon.

In another embodiment, CCF is determined by colonoscopy finding of dilated colon segments, decreased or absent motility of the colon in various segments of the colon, and the altered blood flow pattern of the colonic wall and mucosa.

In one embodiment, the detection of the fungal infection is done by culturing the colonic sample or histochemical analysis of the colonic sample.

In one embodiment, at least one further test is done to detect fungal strains selected from the group consisting of Candida, Saccharomyces, Cryptococcus, and yeast of Malassezia spp.

In one embodiment, the lipase and/or amylase from the subject are further isolated and sequenced for identifying the lipase and/or amylase isoenzyme.

In an embodiment, the method further comprises the step of analyzing the fungal strain to fungal strain infecting the subject's colon.

In an embodiment, the method is used for differentiating acute pancreatitis from CCF.

In another embodiment, the present method may be, used for differentiating acute pancreatitis from CCF, wherein the subject presents at least one symptom associated with Irritable Bowel Syndrome, Ulcerative Colitis, Crohn's disease, or Gastroparesis, Reflux, and a myriad of conditions that include, but are not limited to the symptoms including colonic conditions constipation, diarrhea alternating with constipation, diarrhea predominant IBS or irritable bowel syndrome, inflammatory bowel disease, diverticulitis, diverticulosis, overflow diarrhea, slow transit constipation and anorectal conditions including anal fissure, hemorrhoids, and fistula in ano.

Effects of CCF on the GI tract that occur outside the colon include Slow Gall Bladder leading to voluntary distention which is commonly confused with right upper quadrant (RUQ) pain related to the colon leading to removal of the gallbladder or cholecystectomy, RUQ pain attributed to biliary pain, gastroesophageal reflux disease, dysphagia, aerophagia or swallowing of air with associated distention of the abdomen gas and bloating, and gastroparesis.

Systemic conditions including anorexia, early satiety, hypertension, lymphedema, leaky gut syndrome, leaky vessel syndrome, obesity, and diabetes.

In one embodiment, the level of lipase and/or amylase in the serum of subjects with CCF is increased at least 3-fold compared to control.

In one embodiment, the level of lipase and/or amylase in the serum of subjects is increased 7-10-fold as compared to control.

In one embodiment, the level of lipase and/or amylase in the serum of subjects is increased 2-10-fold as compared to control, wherein the patient does not have pancreatitis when CT scan is done.

In one embodiment, higher lipase levels in the patient are correlated with infection by fungi with higher pathogenicity.

One embodiment of the disclosure is a method of treating CCF by flushing out the colon to remove fecal matter, is followed by a drop in lipase and/or amylase levels confirms the determination of CCF.

In another embodiment of the disclosure flushing out the colon to remove fecal matter can be done by oral laxatives.

In another embodiment of the disclosure flushing out the colon to remove fecal matter can be done by the way of enema.

In one embodiment, the treatment further comprises the step of treating with a pharmaceutical antifungal composition.

In one embodiment, the antifungal composition is selected based on the fungal strain responsible for the fungal infection in the colon.

In one embodiment, the antifungal composition is selected from the group consisting of Amphotericin B, Abelcet, AmBisome, Anidulafungin Eraxis, Butenafine Mentax, Butoconazole Gynazole-1, Caspofungin Cancidas, Clotrimazole Generic, Lotrimin, Mycelex, Econazole Generic, Ecoza, Fluconazole Generic, Diflucan, Flucytosine Generic, Ancobon, Griseofulvin, Grifulvin, Gris-Peg, Itraconazole Generic, Sporanox, Onmel, Ketoconazole Generic, Nizoral, Micafungin Mycamine, Miconazole Generic, Oravig, Micatin, Naftifine, Naftin, Natamycin Natacyn, Nystatin Generic, Oxiconazole Generic, Oxistat, Posaconazole Noxafil, Sulconazole Exelderm, Terbinafine Generic, Lamisil, Terconazole Generic, Terazol, Terazol, Tioconazole Vagistat-1, Monistat Tolnaftate Generic, Aftate, Tinactin, Voriconazole Generic, and Vfend.

In one embodiment, Amphotericin B is used as it is poorly absorbed from the gastrointestinal tract. Oral amphotericin B is thus effective only on fungi within the lumen of the tract.

In one embodiment, a poorly absorbed antifungal agent is used and is effective only on fungi within the lumen of the tract.

In another embodiment of the present disclosure a kit to diagnose CCF is provided. The kit comprising at least a first reagent to identify a fungal species, at least a second reagent to identify fungal enzymes, and a test strip.

In yet another embodiment, the test strip in the kit is made of a membrane adhesively attached to a solid base with the first and the second reagents immobilized on it.

In another embodiment of the disclosure is a diagnostic kit for diagnosing CCF, wherein the kit comprises of at least one reagent to detect fungal amylase and/or lipase from a sample derived from a subject.

In another embodiment, the reagent to detect fungal amylase and/or lipase is an antibody specific to fungal amylase and/or lipase.

In one embodiment, the reagent to detect fungal amylase and/or lipase is a biochemical reagent that undergoes a biochemical reaction to produce a detectable product on reacting with the fungal amylase and/or lipase or at least one peptide derived from the fungal amylase and/or lipase.

In one embodiment, the biochemical reaction is a colorimetric reaction.

In one embodiment, the biochemical reaction is a fluorescent reaction.

In one embodiment, the biochemical reaction is a photometric reaction.

In one embodiment, the kit further comprises a reagent to degrade the fungal amylase and/or lipase into smaller peptides.

In one embodiment, the sample derived from the subject is serum, blood, urine, colonic sample, or stool.

In one embodiment, the kit generates a qualitative result for presence of fungal amylase and/or lipase in the subject's sample.

In one embodiment, the kit generates a quantitative result for presence of fungal amylase and/or lipase in the subject's sample.

In one embodiment, the kit further comprises at least one reagent to identify the fungal strain producing the fungal amylase and/or lipase in the subject's sample.

In one embodiment, at least one reagent to identify the fungal strain identifies fungal strain selected from the group consisting of Candida, Saccharomyces, Cryptococcus and yeast of Malassezia spp.

In an embodiment of the present disclosure, a method (200) to differentiate between congestive colon failure and pancreatitis in a subject is provided.

The method (200) involves evaluating (202) an origin of a lipase from a bodily fluid of the subject, imaging (204) a pancreas and a colon and differentiating (206) between pancreatitis and congestive colon failure based on initial two steps.

In one embodiment, the present disclosure encompasses a method to differentiate between congestive colon failure (CCF) and acute pancreatitis.

In another embodiment, CCF as described herein also encompasses, and/or may result in, may be a causative factor for, or may be associated with, other clinical conditions. Examples of such other clinical conditions include, but are not limited to colonic conditions, effects of CCF on the GI tract that occur outside the colon and systemic conditions like hypertension.

In another embodiment, examples of such colonic conditions include colonic retention of stool, and various conditions are attributed to colon with this stool retention. These colonic conditions include, without limitation, irritable bowel syndrome (IBS), inflammatory bowel disease (IBD), right sided colonic pain, rectal stool impaction, alternating diarrhea and constipation, diarrhea and overflow diarrhea, anal leakage, constipation (right and left sided constipation) and various other named conditions defined as syndromes but with unexplained or poorly explained etiologies. In one embodiment, this also encompasses colonic conditions where the syndrome is well defined, but there are multiple exceptions which have no known treatment or explanation. In one embodiment, these colonic conditions include unique conditions/combination of symptoms, where no satisfactory diagnosis is provided for the symptoms. In such cases, treatments are many times only symptomatic and do not fix the underlying problem making them a lifelong condition. In one embodiment, diagnosing, and treating such symptoms encompasses diagnosing and treating CCF by the methods and compositions described herein.

Right sided colonic pain is a condition where the patient presents with right-sided abdominal pain and various conditions are diagnosed based on location of the pain. In one embodiment of the present disclosure, right upper quadrant (RUQ) pain may be misdiagnosed as cholecystitis. In one embodiment of the present disclosure, right lower quadrant (RLQ) pain may be misdiagnosed as appendicitis.

Right upper quadrant pain may be misdiagnosed as chronic cholecystitis and may include symptoms such as dilated gallbladder due to generalized dysfunction of the gastrointestinal system in CCF, in this case, post cholecystectomy diarrhea leads to correction of CCF and hence improvement, generally lasts for six months after which CCF and symptoms can come back.

In one embodiment, the present disclosure encompasses a method of diagnosing the symptoms including, but not limited to, RLQ pain, RUQ pain, diarrhea, irritable bowel syndrome, Crohn's disease, inflammatory bowel disease (IBD), right sided colonic pain, rectal stool compaction, alternating diarrhea and constipation, diarrhea and overflow diarrhea, anal leakage, constipation (right and left sided constipation), as CCF.

Dilated GB due to CCF will be found in many patients and will be removed. Diarrhea is a common complication of cholecystectomy, and it corrects the symptoms for six months or so after which the symptoms return to baseline.

In another embodiment of the present disclosure, RLQ pain is misdiagnosed as appendicitis. In one embodiment of the present disclosure, RLQ pain is due to CCF.

In one embodiment, symptoms normally associated with IBD or inflammatory bowel disease are due to CCF. In one embodiment, these symptoms are diagnosed and treated using the methods and compositions disclosed herein.

In one embodiment, IBD like symptoms caused by CCF may be misdiagnosed, or insufficiently diagnosed as being due to Crohn's disease, or ulcerative colitis (UC).

In one embodiment, fungal infection associated with CCF may be misdiagnosed as being associated with Crohn's disease. Anti-Saccharomyces cerevisiae antibodies (ASCA) are a specific but only moderately sensitive diagnostic marker for Crohn's disease. In one embodiment, the presence of ASCA antibodies is a prognostic marker for CCF.

In another embodiment, Rub and denudation of colonic barrier defenses is a cause and/or, symptom of CCF. In one embodiment, Rub and denudation of colonic barrier defenses may lead to Crohn's and/or CCF in a subject.

In another embodiment, the damage to the GI tract is caused by the gradual reduction of glycocalyx from the surface mucosa cells. The lack of glycocalyx leads to the entry of foreign substances that lead to an immune response and cause diseases like celiac disease and ASCA positive in Crohn's disease.

Various GI disorders have break in the integrity of cellular defenses due to damaged glycocalyx from the surface mucosa cells leading to diseases. These diseases include colitis, celiac disease with passage of antigens into the deeper layers. Ulcers in the GI tract. Avoiding further injury and providing time for these conditions to heal the mucosa by preventing further damage are well known at this time. However, no therapy is currently in use which can enhance the healing of the mucosa

In another embodiment, CCF leads to, and is also exacerbated by fungal infection. Fungal invasion is associated with CCF, since due to CCF, the mucosal surface is damaged or the defense barrier due to the mucosal surface is compromised.

In another embodiment, this leads to invasion of fungus in the superficial tissue and fungal hyphae which can get close to blood vessels, leaks lipase/amylase into the blood vessels, which leads to the elevated levels of lipase and/or amylase in the bloodstream and also in extra-colonic tissues in the body. Fungal hyphae cannot enter the blood vessels due to body's defenses in immunocompetent patients. Invasion of fungus leads to inflammation that leads to pain. However, in immunocompromised patients that can lead to systemic infection by both bacterial invasion and fungal invasion.

In another embodiment, CCF may be misdiagnosed as UC in some cases.

In one embodiment, CCF leads to constipation, and/or constipation alternating with diarrhea.

Constipation can be right or left sided constipation. Usually, constipation is not diagnosed as right or left sided constipation but as a single entity constipation. This leads to misdiagnosis and mistreatment of specific type of constipation. In one embodiment, constipation can be classified into two different types of constipation. The first type is mainly right sided and presents with anorexia, right lower quadrant/right upper quadrant abdominal pain, nausea vomiting, epigastric pain, weight loss, and abdominal distention. Treatment is difficult and often present with diarrhea alternating with constipation. Fluid and electrolyte abnormalities are also noted.

In another embodiment, liquid laxative is not good for right sided constipation. Laxatives work by enhancing volume and the liquid (added to colon because of the laxatives) just pass by the side of the hard stool, and do not relieve right sided constipation. This has implications for colonoscopy preparation in these patients since this leads to difficulty in cleaning the colon for the colonoscopy procedure. In one embodiment, increased fiber in isolation can lead to worsening of symptoms by increasing the right sided distention of the colon.

In an embodiment, fiber is administered to ‘fluff’ up the colon followed by administration of osmotic laxative.

In yet another embodiment, a particulate laxative loaded with well hydrated small particles is used to stimulate the removal of solid stool from the right side of the colon.

In yet another embodiment, the particulate laxative can be isabgol.

In yet another embodiment, the particulate laxative can be chia seeds.

In yet another embodiment, the particulate laxative can be followed by a liquid laxative to clear the right-side stool.

In yet another embodiment, increased fiber in isolation should not be given for the treatment of CCF.

In yet another embodiment, lactulose is an effective treatment for CCF caused by or causing right sided constipation. Lactulose is an effective laxative for the retained stool on the right side of the colon, since with stool retention, there is an increased load of bacteria which break down the lactulose rapidly by fermentation leading to the release of osmotic load and gases which cause the removal of stool from the right colon. It is a scientific fact that the motility in the colon proximal to the hepatic flexure is towards the cecum and distal to the hepatic flexure is towards the rectum. This helps in compacting the stool and allowing the right side of the colon to continue to absorb water until the liquid stool has turned into semisolid to solid stool which is then passed beyond hepatic flexure. In one embodiment, treatment of right-sided stool retention with liquid laxatives (like polyethylene glycol or PEG) which increase the volume by adding liquids slowly, do not work well for the treatment of CCF, and these laxatives can lead to diarrhea and further electrolyte imbalances in patients suffering from CCF.

In yet another embodiment, lactulose works well in such conditions as the small amount gets around the stool, is broken up by the bacteria. Due to osmotic effect of multiple small fragments of lactulose, it sucks water into the colon causing significant volume in the right colon causing the stool to be flushed from the right side of the colon. In one embodiment, lactulose gets to the nucleus of the stool ball and starts breaking it up into smaller segments which is easy to pass from the right side of the colon. However, repeated use of lactulose over time can lead to resistance and no effect of lactulose is found in some patients. In one embodiment, these patients benefit from particulate laxatives like isabgol and Chia seeds.

In yet another embodiment, the present disclosure encompasses methods and compositions to use laxatives for treatment of CCF.

In yet another embodiment, CCF patient presents with symptoms of diarrhea and/or overflow diarrhea: sometimes a large single ball forms in the rectum, which is unable to pass, and the colon will start secreting water around the ball which keeps on leaking out from the rectum leading to overflow diarrhea and soilage. This leads to electrolyte imbalance. This can be treated by stool disimpaction.

In yet another embodiment, CCF presents with alternating diarrhea and constipation as a result of CCF: many of the patients have constipation alternating with corrective diarrhea which happens because the stool hardens and since significant constipation is encountered, the body reverses absorption of fluid and start secreting fluid to dissolve the hard stool. The capability is only available on the left side of the colon. Patients generally pass liquid stool with hard balls of stool. Usually, they feel better for a few days after an episode of passing diarrhea with hard balls of stool and then the cycle repeats.

In yet another embodiment, CCF patients present with rectal stool impaction: as multiple stool balls that compressed together, they form one large ball which the person is unable to pass through the anus. This leads to impaction and further complication. If the person forces these hard balls out it causes trauma to the anorectal area leading to rectal bleeding.

In yet another embodiment, CCF may be misdiagnosed as Idiopathic Constipation syndrome (ICS), due to mismanaged constipation.

The present disclosure also encompasses diagnosis of symptoms associated with effects of CCF that occur outside the colon. Examples of such outside colon effects and/or their symptoms are given below, without limitation.

Preload is defined in the context of congestive heart failure as excessive blood that need to be pumped by the heart and afterload is defined in the context of congestive heart failure as the increased resistance against which the heart will have to pump the blood. In congestive colon failure preload is defined as excessive stool in the colon, which compromises the function of the colon by causing the dilation of the colon leading to inefficient contractions. Afterload is defined as obstructed stool in the distal part of the colon which increases the force required to move the stool in the colon. Both preload and afterload lead to ineffective colonic function and lead to congestive colon failure.

In yet another embodiment, a two-step treatment for CCF is proposed, with preload and afterload concept (adapted from congestive heart failure), is used for better communication to the patient and understanding by the patient in a better way. Step one involves the acute flushing of the stool from the right side of the colon (preventing the preload). Step two involves preventing constipated retention of stool in the colon (preventing the afterload).

In yet another embodiment, CCF leads to esophageal symptoms. The acid mixed food in the stomach is regurgitated back into the esophagus causing reflux symptoms. In one embodiment, one of the effects of CCF outside the colon is gastroparesis. Since in CCF, the stool in the colon is not moving forward and distends the colon it provides feedback inhibition to the stomach to hold the food leading to gastroparesis.

In yet another embodiment, one effect of CCF outside the colon is on the gall bladder (GB). Dilated GB due to CCF: since the feedback inhibition paralyzes the gut it also includes the smooth muscle of the gall bladder. This leads to paralysis of the gall better leading to distention and stone formation because of stasis. In one embodiment, a patient who is presenting with right-sided abdominal pain and evaluated for gallbladder removal although it is not dysfunctional or diseased.

Nonalcoholic Steatohepatitis (NASH): Nonalcoholic fatty liver disease (NAFLD), is the hepatic manifestation of a metabolic syndrome, and is the most common cause of elevated liver enzymes in the United States. NAFLD with inflammation and fibrosis is known as Nonalcoholic steatohepatitis (NASH) because it resembles alcoholic liver disease (ALD) without a history of alcohol ingestion.

In yet another embodiment, symptoms similar to NASH may be found as a result of CCF. With stool retention increased colonic products are carried by the portal vein towards the liver, leading to inflammation of the liver, long-term damage, and cirrhosis.

In another embodiment, treatment of CCF can lead to the improvement of Nonalcoholic steatohepatitis (NASH).

In another embodiment, one effect of CCF outside the colon is on the small bowel.

In another embodiment, one of the effects on small bowel due to CCF is bacterial overgrowth: with stool retention the distal small bowel is also backed up and loaded with bacterial content. Because of stasis and paralysis of the small bowel various forms of bacterial overgrowth and associated symptoms develop.

In another embodiment, one of the effects on the small bowel due to CCF is development of celiac diseases. There are many unknown facts in celiac disease. In another embodiment, damage to the small bowel or large bowel due to CCF leads to increased absorption of gluten leading to systemic reaction and gluten sensitivity. Celiac disease (CD) is characterized by villous atrophy with crypt hyperplasia and inflammation of the small intestinal mucosa leading to disturbed epithelial transport. In untreated CD, fat malabsorption can occur. The colonic mucosa does not get adequate amount of lipids and glycocalyx that stimulates the growth of colonic mucosa. The restriction of fat in the diet for weight loss is a contributory factor to gluten intolerance and celiac disease.

In another embodiment, providing adequate lipid to the colon/bowel can help in treatment/healing of mucosal surfaces.

In another embodiment, the present disclosure encompasses use of nutrients to repair and replace the scarce resources lost in the gut due to recurrent damage, malabsorption, or due to insufficient intake. In absence of sufficient absorption, and/or intake of nutrients, deficiency spreads and causes systemic damage.

In yet another embodiment, CCF and/or damage to the colon or small bowel due to CCF can be treated by intake of lipids, glycocalyx composition or a combination thereof.

It is a well-known fact that free fatty acids are trophic to the colonocytes and help in the healing of the colon however with fat intake majority of fat is absorbed in the small bowel leading to obesity and deposit of fat in the liver which creates an environment for second hit Hypothesis of NASH.

In another embodiment, the NASH patients are at elevated risk for further developing CCF. As the fat is staying longer in the small bowel due to decreased motility of the G.I. tract, increased absorption is taking place leading to increased deposit of fat in the liver causing fatty liver. The increased fat in the body can lead to Obesity. Majority of patients will then decrease the intake of fat significantly for physical appearance, further leading to precipitation of congestive colon failure and worsening of symptoms.

In one embodiment, the present disclosure encompasses a method to treat CCF, the method comprising the step of using fatty acids/fats to heal the colon. In one embodiment, the method further comprises the step of administering fatty acids to the subject, and the step of preventing the absorption of fats subsequently in the subject's gut.

In one embodiment, the present disclosure encompasses the following steps for reducing the absorption of fats, as a step of healing the colon by administering fatty acids to the subject consists of adding a lipase inhibitor to a diet prior to the intake of fat, providing free fatty acids and granules or other forms of delayed absorption and administering a colonic enema where the fat is travels all the way up to the cecum

In an embodiment, granules of lipase inhibitor have a pH dependent mechanism where the change in the pH will trigger the dissolution of membrane of the granule

In another embodiment, granules of lipase inhibitor have a time-based release mechanism.

With the release of FFA (Free fatty Acids) directly in the colon, healing of the mucosa can take place.

In one embodiment, one of the systemic cardiovascular effect due to CCF is hypertension, due to distention of colonic lumen, the pressure at mucosa increases leading to decrease in the surface perfusion of the colonic mucosa causing hypoxia. This hypoxia stimulates body by mechanisms unknown to increase blood pressure to correct mucosal perfusion, leading to hypertension. It is a well-known fact that constipation and any hollow organ distention leads to increase in blood pressure. Ischemia to the brain at the time of embolic stroke leads to hypertension, in an attempt to correct low perfusion of brain tissue. Similarly, renal artery stenosis leads to hypertension. Although the mechanisms are ill-defined, the assumption that mucosal ischemia will not lead to similar systemic corrective response appears erroneous.

In one embodiment, suspicion of CCF helps the diagnosis of CCF, along with the established principles of diagnosing, monitoring, and treating congestive heart failure (CHF).

In one embodiment, timely diagnosis of CCF helps to decide on the treatment of CCF, along with the established principles of diagnosing, monitoring, and treating congestive heart failure (CHF). In an embodiment, preload can be reduced by fasting or providing a low residue diet. in an embodiment, afterload can be reduced by laxatives.

In one embodiment, laxatives are used for treating CCF even when the patient is having diarrhea. In one embodiment, the cause of diarrhea in such patients is overflow diarrhea. In one embodiment, increasing fiber in diet to treat constipation treats episodic left sided colonic constipation, but is harmful in right sided colonic constipation as described above.

In one embodiment, timely diagnosis of CCF helps to decide on the monitoring of CCF, along with the established principles of diagnosing, monitoring, and treating congestive heart failure (CHF).

In one embodiment, one of the effects on the GI tract due to CCF is, the disruption of water salt balance in the body. In one embodiment, correcting the water salt balance in the body may be an effect and/or a method of treatment of CCF in the body.

In one embodiment, the present disclosure encompasses Diagnostic testing for CCF comprising fungal lipase testing, chronic pancreatitis diagnostic test and stool movement in colon.

In an embodiment of the present disclosure, a method (300) to detect congestive colon failure in a subject is provided.

The method (300) includes administering (302) a set of sensors in the subject through the subject's mouth, tracking (304) a movement of the set of sensors through a gastro-intestinal tract of the subject, mapping (306) the movement of each sensor over time to compile a composite map of a movement of stool over time and detecting (308) congestive colon failure based on the composite map.

In another embodiment the stool movement in the method (300) is tracked using a radio frequency identification (RFID) sensor.

In another embodiment the method (300), wherein the stool movement is tracked by the way of tracking a movement of a radio frequency identification (RFID) sensor inside a digestive tract.

In another embodiment, CCF is caused by disbalance of commensal microorganisms in the gut. In one embodiment, the disbalance is by depletion of anaerobic commensal gut microorganisms. In one embodiment the disclosure encompasses a method of treating CCF by augmenting commensal anaerobe microorganism population in the gut by consuming fermented foods rich in anaerobic microorganism. In one embodiment, the process of making fermented foods rich in anaerobic microorganism involves moderate cooking followed by time-based fermentation in anaerobic conditions by adding a layer of oil during the process to create an anaerobic environment.

In another embodiment, the strength of anaerobic culture is communicated to the patient by labelling the product with anaerobic index. This index will certify the strength of anaerobic organisms present in the product.

In another embodiment, a hygroscopic sensor is used for the detection of constipation. In one embodiment, a major causative factor for constipation is lack of water in the colon.

In an embodiment, fiber is administered to ‘fluff’ up the colon followed by administration of osmotic laxative.

In another embodiment, a hygroscopic sensor is ingested by the patient. In yet another embodiment, the hygroscopic sensor has a battery and sends a signal to a receiver close to the body of the patient to detect the state of water in the stool. In yet another embodiment, the sensor is without a battery and is used as a passive reading device like the passive RFID device. In yet another embodiment, the sensor detects the salt content and the water content of the stool or a ratio thereof. In another embodiment, the sensor is retrieved. In another embodiment, the hygroscopic sensor is used to detect water content in a subject's stool. The lack of water as indicated by the hygroscopic sensor can be used to predict constipated fecal discharge. In another embodiment, a hygroscopic sensor is used for detection of constipation, and hence as a diagnostic tool for detection of CCF.

In another embodiment, the commensal anaerobic microorganisms are beneficial in treatment of CCF since they produce significant amounts of lipids which are therapeutic for the use of healing the colonic mucosa. In one embodiment, CCF can lead to harmful microorganism overpopulation in the small bowel. In one embodiment, the commensal anaerobic microorganisms are beneficial in treatment of CCF since they produce significant amounts of lipids which are therapeutic for the use of healing the small bowel mucosa. In a preferred embodiment, a method for treating CCF by administering an effective amount of anaerobic microbial culture is provided. In a preferred embodiment, the anaerobic microbial culture includes a fermented food. In a preferred embodiment, the anaerobic microbial culture includes bacterial, yeast, fungal or any other microbial anaerobic culture. In another preferred embodiment, the anaerobic microorganism culture includes pickled vegetables, fruits, and meats.

In a preferred embodiment, the presence of a significant amount of lipids in an anaerobic bacterial culture enables healing of colonic mucosa.

In another embodiment, RFID chips are used to detect constipation.

Constipation can be described by multiple terms including but not limited to constipation, slow transit constipation, obstructive constipation, neurogenic constipation, drug induced constipation, narcotic pain medication induced constipation, episodic constipation, chronic constipation, normal-transit, slow-transit and pelvic floor dysfunction. irritable bowel syndrome constipation, predominant IBS-C; primary constipation, secondly constipation.

Other descriptions of constipation include Organic constipation due to Colorectal cancer, extra-intestinal mass, post inflammatory, ischemic, or surgical stenosis. Endocrine or metabolic induced constipation due to diabetes mellitus, hypothyroidism, hypercalcemia, porphyria, chronic renal insufficiency, panhypopituitarism, pregnancy. Neurological induced constipation due to Spinal cord injury, Parkinson's disease, paraplegia, multiple sclerosis, autonomic neuropathy, Hirschsprung's disease, chronic intestinal pseudo-obstruction. Myogenic induced constipation due to Myotonic dystrophy, dermatomyositis, scleroderma, amyloidosis, chronic intestinal pseudo-obstruction. Anorectal disease induced constipation due to Anal fissure, anal strictures, inflammatory bowel disease, proctitis. Drugs induced constipation due to Opiates, antihypertensive agents, tricyclic antidepressants, iron preparations, anti-epileptic drugs, anti-Parkinsonian agents (anticholinergic or dopaminergic). Diet or lifestyle induced constipation due to low fiber diet, dehydration, inactive lifestyle.

In one embodiment, active RFID sensors are ingested by the patient.

In one embodiment, passive RFID sensors are ingested by the patient.

In one embodiment, a single RFID sensor is ingested by the patient.

In one embodiment, a plurality of sensors are ingested by the patient.

In one embodiment, RFID chips are used to detect CCF in adult patients/subjects with severe constipation but who otherwise present negative GI evaluations for other disorders.

In one embodiment, RFID sensors are used to replace SITZ marker study.

In another embodiment, a method of detection of CCF using RFID is provided. In one embodiment, orally administered capsule(s) with multiple small RFID sensors are given to the patient. The capsule disintegrates in the G.I. tract, thus releasing multiple RFID sensors in the G.I. tract. The movement of the administered RFID sensors is monitored as it passes through the GI tract to determine their presence and path in the GI tract at all times, they are present in the GI tract. In one embodiment, all RFIDs can be tracked individually, recovering the data from the movement of each sensor, can help put a composite map together with the movement of the sensors, and a time-based composite map of individual sensors over time is built, and in one embodiment, connecting the above two maps to give a time-based map of the stool flow.

In another embodiment, RFID sensors are embedded in a subject's meal and is used to determine small bowel bacterial overgrowth. Location of the RFID sensor along with the hydrogen peak in the breath test confirms the presence of bacterial overgrowth.

In an embodiment, RFID sensor is administered with a sugar that the human body cannot metabolize. In another embodiment the sugar is lactulose.

The above-mentioned method has the advantages of not using radiation, and no additional hospital visits, since data is collected by the RFID sensor reader via GPS like tracking using the RFID reader, wherein the RFID chips are used like a capsule endoscope. In one embodiment, this data provides additional parameters and characteristics of the path, presence, pace (e.g., with which the RFID sensor travels within the G.I. tract in different patients) and several other such insights to make a diagnosis related to GI disorder. In one of the embodiments, the transition of sensor/sensors in the body provides real-time tracking instead of comparing different images of the tract obtained over a duration of time such as in an accepted protocol three X-ray images over a week are used to determine or diagnose chronic constipation or even predict the path of the capsules within the GI tract. Real-time tracking gives better diagnosis and more insights into various G.I. disorders including but not limited to the colonic problems and congestive colon failure. In one of the embodiments, the passage of sensor in the small bowel is recorded to provide the diagnosis of small bowel motility abnormalities. In one of the embodiments, the passage of sensors in this, is recorded to provide the diagnosis of gastroparesis. In one of the embodiments, the regurgitation of the sensors from the stomach into the esophagus is used as a diagnosis of reflux.

In an embodiment, a method to differentiate a ‘real’ diarrhea from an overflow diarrhea is provided. In overflow diarrhea, the stool passes by the side of the hard impacted stool balls and exits out of the rectum. Patients may, therefore, sometimes report rapid transition of the ingested food matter through the GI tract. This overflow diarrhea may give a false negative [with respect to CCF] or positive [with respect to “real diarrhea] and therefore has to be differentiated from a real diarrhea to enable accurate diagnosis, prognosis and therapy of the condition. The real diarrhea is the one in which the rapid transit is because of pathological reason and/or malabsorption or other conditions like infectious problems which cause malabsorption or increased secretion of fluids.

In an embodiment, a method of differentiating overflow from real diarrhea includes administering capsules, to a patient, containing food colors such that different colors are administered within a fixed time frame.

In an embodiment, a blue color, food color capsule is given to the patient in the morning, fluorescent yellow color is given the next day morning. If the colon is healthy and functioning properly, the second day stool color will be blue and the third day stool color will be fluorescent yellow. However, in case there is fragmentation of the stool with some components being passed early on and some being retained the color of stool may turn into green and a noticeable color will continue over next few days.

This color change can be picked up by images taken of stool over successive days. The process can be automated using electronic sensors embedded in the commode. Artificial intelligence tools can be used to differentiate the stool images.

In one embodiment, a method of treatment for CCF comprises the step of administering non-absorbable lipids/fats to the subject/patient.

In one of the embodiments, lipase inhibitor orlistat is used, this inhibits the lipase enzyme, thus preventing digestion of fat, thus preventing absorption of fat, thus increasing the delivery of fat to the colon. In one of the embodiments, fat can be encased in a time release capsule, delayed-release capsule, pH dependent release mechanism or by other mechanisms to provide release of fat directly into the colon. If the fat is released in the small bowel it leads to absorption of the fat.

In one embodiment, the non-absorbable fat is from Butter fish in portions that do not cause GI discomfort. In one embodiment, the nonabsorbable lipids are wax esters, which are poorly absorbed by humans. In one embodiment, the nonabsorbable lipids are used as a treatment for CCF.

Constipation is a poorly understood subject. Various substances have been tried to treat constipation and to help the colon like laxatives, dietary changes like fiber, increasing water intake, Castor oil for constipation to increase lubrication. Most of them will work for a short time and then fail. Thus, leading to constipation as a major problem throughout the world. The health of colonic mucosa is dependent on free fatty acids. Short chain fatty acids are required for the health of colocytes which are the cells lining the colon.

In another embodiment, the method of treating CCF excludes the step of administering laxatives to the subject/patient. In one embodiment, administering fat into the colon, by oral or by some other means, helps to heal the colonic mucosa. However, adding fat by dietary intake may not be very effective, since most of the fat is absorbed by the small bowel leading to obesity and other related health consequences.

In one embodiment, the fat administered to the subject for treatment of CCF is not absorbed by the small bowel but is rapidly delivered into the colon. In one embodiment, free fatty acids are produced naturally in the colon by the effect of bacteria/probiotics on the prebiotics taken orally by the subject.

In yet another embodiment, the delivery of fat in the colon leads to satiety and decreases calorie intake and is used for treating obesity.

In another embodiment, prebiotics, without limitation, include lactulose, sucrose, sucralose, mannose and other components which promote the growth of good microorganisms. In one embodiment, an additional impact of congestive colon failure on the amount of fat in the colon is due to the delayed emptying of the small bowel, which causes the food to stay longer in the small bowel, leading to continued absorption of digested material and fat is also absorbed in a more efficient manner in the small bowel. This leads to minimal, or no fat left in the digested chyme reaching the colon, which is part of the digested food in the small bowel.

In another embodiment, to increase the amount of fat delivered to the colon, following methods are used, which are encompassed by the present disclosure.

Fat is digested by the action of lipase and bile acids on the fat globules to increase emulsification and breakdown the fat into smaller components to increase the absorption of fat. In one embodiment, lipase inhibitors like orlistat are used to prevent the breakdown of triglycerides into components which can be absorbed, in the CCF patients. Thus, this medication can increase the delivery of fat to the colon.

In another embodiment, the method comprises the step of increasing the delivery of fat to the colon by encasement of the fatty acids by pharmaceutically acceptable carriers, and the encapsulated fats are delivered into the colon based on delayed release or time release or pH-based release of the fatty acids in the colon. In one embodiment, this is done by pharmacological coating.

In another embodiment, prebiotics and/or probiotics are administered to the subject, by material in colonic delivery systems. This will prevent the absorption of such material in the small bowel.

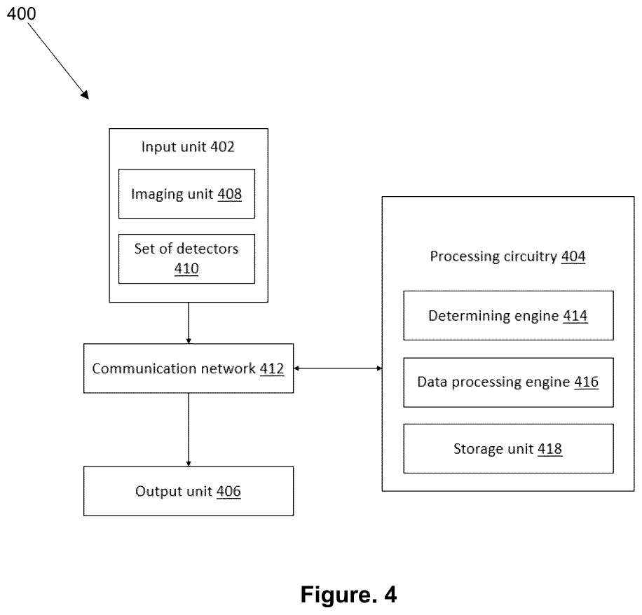

FIG. 4 illustrates a system 400 (hereinafter referred to and designated as “the system”), according to an embodiment herein.

In another embodiment of the present disclosure, a toilet system is provided.

The toilet system (400) includes an input unit (402) configured to determine a set of attributes associated with a human waste and generate a first signal based on the set of attributes of the human waste. A processing circuitry (404) coupled with the input unit (402) and configured to receive the first signal from the input unit and analyze the first signal to determine an intestinal health of the subject. An output unit (406) coupled with the processing circuitry (404) and configured to relay the gastro-intestinal health of the subject on an audio-visual device of the output unit (406) and interact with users to receive instructions from the user.

In another embodiment of the toilet system (400) of the present disclosure, the input unit consists of an imaging unit (408) configured to capture images, a set of detectors (410) configured to detect signals representing temperature, pressure, weight, texture, color, consistency, flow or a combination thereof, and an audio unit configured to receive sound. The set of detectors 410, the processing circuitry 404 and the output unit 406 may be communicatively coupled to each other via the communication network 412. In some other aspects of the present disclosure, the set of detectors 410 and the processing circuitry 404 may be communicably coupled through separate communication networks established there between. In some aspects of the present disclosure, the communication interface may be one of, but are not limited to, a modem, a network interface such as an Ethernet card, a communication port, and/or a Personal Computer Memory Card International Association (PCMCIA) slot and card, an antenna, a radio frequency (RF) transceiver, one or more amplifiers, a tuner, one or more oscillators, a digital signal processor, a coder-decoder (CODEC) chipset, a subscriber identity module (SIM) card, and a local buffer circuit. It will be apparent to a person of ordinary skill in the art that the communication interface may include any device and/or apparatus capable of providing wireless or wired communications between the set of sensors 402 and the processing circuitry 404.

In some aspects of the present disclosure, the processing circuitry 404 may be implemented as one or more microprocessors, microcomputers, microcontrollers, digital signal processors, central processing units, logic circuitries, and/or any devices that manipulate data based on operational instructions stored in the storage unit (not shown). Computer-readable instructions or routines stored in the storage unit may be fetched and executed to create or share data units over a network service. The storage unit may include any non-transitory storage device including, for example, volatile memory such as RAM (Random Access Memory), or non-volatile memory such as EPROM (Erasable Programmable Read-Only Memory), flash memory, and like. Aspects of the present disclosure are intended to include and/or otherwise cover any type of the storage unit, including known, related and later developed technology.

In some aspects of the present disclosure, the processing circuitry 404 may be implemented as a combination of hardware and instructions (for example, machine executable instructions) to implement one or more functionalities of the processing circuitry 404. In examples described herein, such combinations of hardware and instructions may be implemented in several different ways. In an exemplary scenario, the instructions for the processing circuitry 404 may be processor executable instructions stored on a non-transitory machine-readable storage medium and the hardware for the processing circuitry 404 may include a processing resource (for example, one or more processors), to execute such instructions. In such examples, the processing circuitry 404 may include the machine-readable storage medium to store the instructions and the processing resource to execute the instructions, or the machine-readable storage medium may be separate but accessible to the processing circuitry 404 and the processing resource. In other examples, the processing circuitry 404 may be implemented by an electronic circuitry.

The processing circuitry 404 may include a determination engine 414, and a data processing engine 416.

The determination engine 414 may be configured to receive set of attributes from the set of detectors 410 and one or more images of the human waste from the imaging unit 408. In another embodiment of the toilet system of the present disclosure, the set of attributes of the human waste include smell, consistency, texture, color, flow, temperature.

In yet another embodiment the system (400), wherein the imaging unit is equipped with thermal imaging and ultra-sound imaging techniques.

In yet another embodiment the system (400), wherein the pressure sensor is selected from a group comprising potentiometric pressure sensors, inductive pressure sensors, capacitive pressure sensors, piezoelectric pressure sensors, strain gauge pressure sensors, and variable reluctance pressure sensors.

In another embodiment, the set of detectors 410 that includes an audio recorder can be placed close to the defecation site, to record defecation or bowel movement and urination sounds, to predict gut health. It will record the audio during the act of defecation and urination. The audio can be used to differentiate between various forms of stool. It can differentiate diarrhea or liquid stool, hard balls falling into the liquid trap of the commode, passage of gas, or a combination of above.

The determination engine 414 may be configured to differentiate the CCF from other pancreatic and colon associated diseases based on the attributes received from the input unit 402. The determination engine 414 may be configured to compare the set of attributes and one or more images received from the input unit 402 with a prestored threshold attributes and images respectively, stored in a storage unit 418. Such that the determination engine 414 differentiate the CCF from other pancreatic and colon associated diseases.

The data processing engine 416 may further be configured to receive the differentiated data from the determination engine 414 and the set of signals from the set of sensors (consumed orally). The data processing engine 416 may further be configured to generate the composite map of movement of stool overtime. The data processing engine 416 further be configured to confirms the presence of CCF based on the set of signals received from the set of sensors and the differentiated data received from the determination engine 414.

In some aspects of the present disclosure, the data processing engine 416 observe the differentiated data from the determination engine 414 over a period of time and further generates a report based on time series of data.

The data processing engine 416 may further communicate the analyzed data to output unit 406. In another embodiment, an application can help differentiate between different types of stools based on the recording, and artificial intelligence can help in this prediction. In another embodiment, the timing of urination if studied repeatedly over time can predict dehydration in the patient. In another embodiment, the combination of low urination time with hard stool passage can lead to the diagnosis of CCF. In another embodiment, treatment can be recommended based on audio recording. In another embodiment, sitting on the commode seat to activate the recording and document the number of visits to the bathroom. In one embodiment, the proximity sensor of the phone can be used to identify the person sitting on the pot. In another embodiment, a profile of the user is created and linked with the user's phone.

In another embodiment, the recording is activated when the subject sits on a pot seat, and thus there are no privacy issues. In one embodiment, the data is uploaded to the cloud using the person's cell phone. In one embodiment, medical help is provided to the subject based on the bowel movement recording, which is uploaded on the app. In one embodiment, simultaneous recording of urine flow duration and speed also is used for diagnosing and/or detecting medical problems.

In another embodiment, the method of treating CCF comprises the step of cycling fiber and fibre free diets. The underlying problem in congestive colon failure is the excess of fiber and compacted stool in the right side of the colon which prevents the functioning of the colon in an optimal fashion. Excessive intake of significant fiber-rich diet does not help, while the rest of the colon is impacted with desiccated stool at a distal point. Hard stool in the rectum can stimulate the rectal and sigmoid region to secrete liquid causing the person to have liquid stool with solid components while having impacted stool in the proximal part of the colon. These underlying problems if not corrected will continue and the patient will suffer from alternating diarrhea and constipation and other symptoms related to congestive colon failure. In one embodiment, fiber coming into the colon is decreased for some time by either fasting or decreasing the amount of fiber taken in the meals. This provides time for the colon to clear the previously ingested fiber which has turned into solid stool. In one embodiment, taking a fiber free diet or a low fiber diet enhances the probability of completely clearing the colon prior to the next meal.

In another embodiment, a balance between adequate nutrition and low fiber to decompress the colon is maintained to treat CCF.

In another embodiment, a significant advantage of correcting congestive colon failure is the associated weight loss and the feeling of well-being.

In another embodiment, correction of congestive colon failure leads to increased appetite once the colon is clear of any residual stool.

In another embodiment, correction of congestive colon failure leads to correction of refractory reflux or gastroesophageal reflux disease once the colon is cleared of any residual stool.

In another embodiment, correction of congestive colon failure leads to correction of abdominal bloating, distention, gas, or other dyspepsia related symptoms once the colon is clear of any residual stool.

In another embodiment, correction of congestive colon failure leads to correction of abdominal pain and decreases the associated use of narcotic pain medication once the colon is clear of any residual stool.

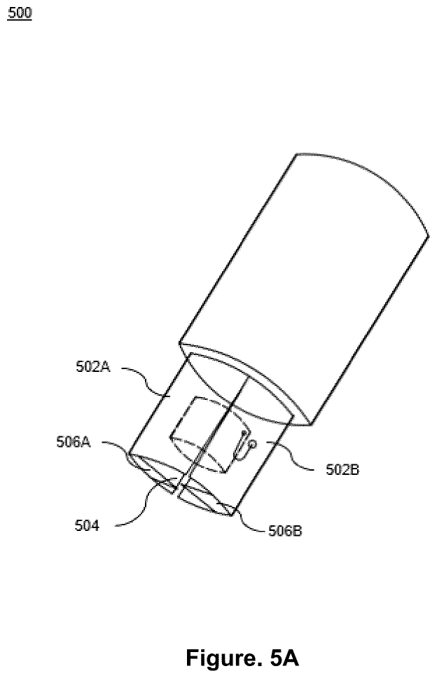

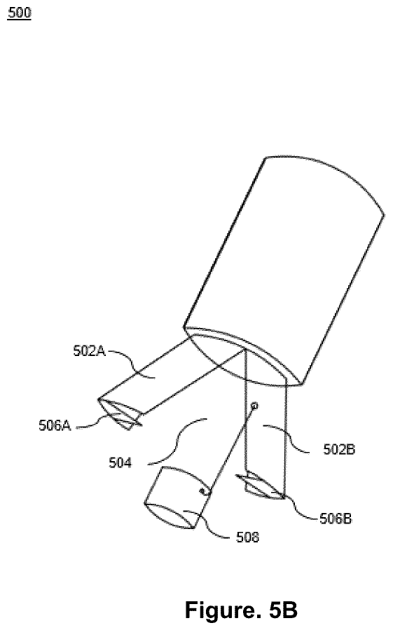

In another embodiment, treating CCF is an important step in treating chronic obesity in a subject. FIGS. 5A and 5B illustrates a clip device 500, according to an aspect herein.

The clip device 500 may be disposed in a food track of the subject by way of endoscopy insertion.

The clip device 500 may include one or more binding portion 502A&502B (of which first through second binding portion is shown) and a carrier portion 504. The number of binding portions are not only limited to two as depicted in FIGS. 5A and 5B, but also include more than two binding portions, wherein the functionality of the binding portion remains on hooking with any surface of interest.

The binding portion 502 may enable the clip device 500 to bind on walls of the food track of the subject.

Each binding portion of the one or more binding portions 502 may include at least one hook 506. The hooks 506 may be disposed on internal side of the corresponding binding portions 502. The hook of a first binding portion 502A may receive a hook of a second binding portion 502B such that, the clip device 500 is clipped to the walls of the food track of the subject.

The carrier portion 504 may include diodes 508.

The diodes 508 may be configured to detect signals representing one or more chemical parameters in the food track of the subject and further communicates with the processing circuitry 404.

In some aspects of the present disclosure, the diodes 508 may be attached to at least one portion of the one or more binding portion by way of a rope. In some aspects of the present disclosure, the rope may be hitched between a first bore provided on the aperture of at least one binding portion 502 and a second bore provided on the diodes 508.

The determination engine 414 in the processing circuitry 404 may be configured to compare the detected signals with a prestored threshold chemical parameters stored in the storage unit 418. The data processing engine 416 may be configured to receive the compared data from the determination engine 414 and further configured to analyze a biological condition of the user.

In an exemplary scenario, the diode 508 may be clipped near duodenum, the diode 508 may include a pH (power oh hydrogen) sensor, that may be configured to sense pH of pancreatic juice secreted by pancreas. The determination engine 414 may be configured to receive detected pH of pancreatic juice from the diodes 508. The determination engine 414 may be configured to compare the received pH with a prestored threshold chemical parameters that include a threshold pH of pancreatic juice stored in the storage unit 418. The data processing engine 416 may be configured to receive the compared data from the determination engine 414 and further be configured to generate results representing the normality and abnormality of pH. The data processing engine 416 may further be configured to communicate the generated results with the user by way of the output unit 406.

In another exemplary scenario, a biological agent may be provided to the user by way of a parenteral route. The biological agent may enhance a flow of the pancreatic juices. In some aspects of the present disclosure, the biological agent may be a secretin that may have high bicarbonate concentration. The diode 508 may be adapted to detect the pH of the pancreatic juices in the duodenum. In some other aspects of the present disclosure, the diode 508 may be adapted to detect a level of bicarbonate in the pancreatic juices. In some other aspects of the present disclosure, the bicarbonate concentration may neutralize an acid generated by the stomach.

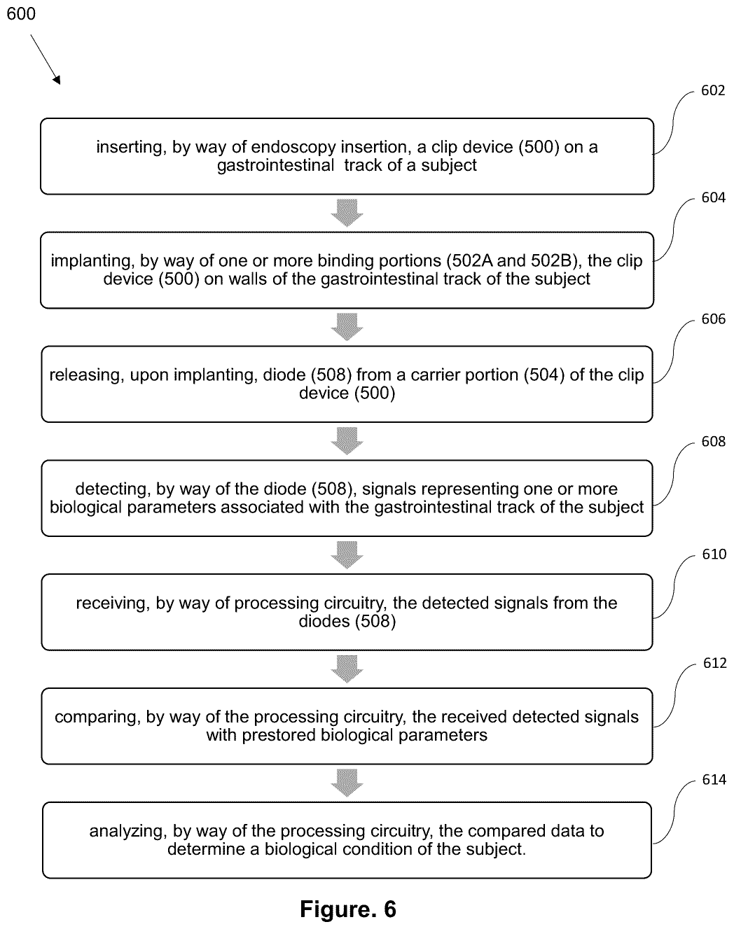

FIG. 6 illustrates a flowchart that depicts a method 600 of detecting Congestive Column Failure, according to an aspect herein. the method includes the following steps:

At step 602, inserting the clip device 500 on a gastrointestinal track of the subject by way of an endoscope. At step 604, implanting the clip device 500 on walls of the gastrointestinal track of the subject by way of one or more binding portions 502A-502B.

At step 606, releasing the diode 508 from the carrier portion 504 of the clip device 500 upon implanting the clip device 500.

At step 608, detecting signals representing the one or more biological parameters associated with the gastrointestinal track of the subject upon releasing the diodes 508. In some aspects of the present disclosure, the biological agent such as secretin may be provided by the parental route to enhance a flow of pancreatic juice in the body of the user. In some other aspects of the present disclosure, the one or more biological parameters include pH of the pancreatic juice.

At step 610, receiving the detected signals from the diodes 508 by way of a processor (not shown). At step 612, comparing the received detected signals with a prestored biological parameter stored in a storage unit by way of the processor.

At step 614, analyzing the compared data to determine the biological condition of the subject by way of the processor.

1. A method (100) of treatment for congestive colon failure (CCF) in a subject, the method comprises:
a. evaluating (102) an origin of a lipase and/or an amylase from a biological fluid sample from the subject;
b. optionally analyzing (104) a level of the lipase and/or an amylase from the biological fluid sample from the subject;
c. optionally imaging (106) a colon and a pancreas of the subject; and
d. providing (108) laxatives, an anti-fungal composition, fat, a composition selected from a group comprising glucosamine, methylsulfonylmethane (MSM) rhamnan sulfate, Sulodexide, Rosuvastatin, Metformin, Hydrocortisone, Antithrombin, Etanercept, Heparin, Albumin, Fresh frozen plasma (FFP), Poloxamer-188, vitamins, trace elements, a locally acting vasodilator, anaerobic microbial culture, pickled fruits, pickled vegetables, preserved meat, and fermented juices or a combination thereof based upon results of step a and optionally from steps b to c.

2. The method (100) of claim 1, wherein origin of the lipase and/or amylase is evaluated by the way of analyzing biomarkers related to lipase and/or amylase.

3. The method (100) of claim 1, wherein imaging is done by the way of magnetic resonance imaging (MRI), X-Ray, CT-scan, ultrasound, thermal imaging, nuclear imaging, physical exam, or a combination thereof.

4. The method (100) of claim 1, wherein the fat is selected from a group comprising non-absorbable fats, wax esters, and fat from butter fish, or a combination thereof.

5. The method (100) of claim 1, further comprises alternating between fiber and fiber free diet wherein fiber diet comprises of dietary and supplemental fiber.

6. A kit to diagnose congestive colon failure (CCF), the kit comprises:
a. at least a first reagent to identify a fungal species;
b. at least a second reagent to identify fungal enzymes; and
c. a test strip made of membrane adhesively attached to a solid base with the first and second reagents immobilized on it.

7. A method (200) to differentiate between congestive colon failure and pancreatitis in a subject, the method comprises:
a. evaluating (202) an origin of a lipase from a bodily fluid of the subject;
b. imaging (204) a pancreas and a colon; and
c. differentiating (206) between pancreatitis and congestive colon failure based on steps a and b.

8. A method (300) to detect congestive colon failure in a subject, the method comprising:
a. Administering (302) a set of sensors in the subject through the subject's mouth;
b. Tracking (304) a movement of the set of sensors through a Gastro-intestinal tract of the subject;
c. mapping (306) the movement of each sensor over time to compile a composite map of a movement of stool over time; and
d. detecting (308) congestive colon failure based on the composite map.

9. The method (300) of claim 8, wherein the stool flow overtime is tracked using a set of Radio Frequency Identification (RFID) sensors.

10. The method (300) of claim 8, wherein the Radio Frequency Identification sensors are hygroscopic and provide real-time information.

11. A toilet system (400) comprises:
a. an input unit (402) configured to detect a set of attributes selected from a group comprising smell, consistency, texture, color, flow, and temperature associated with a human waste and generate a first signal based on the detected set of attributes;
b. processing circuitry (404) coupled with the input unit (402), and configured to:
(i) receive the first signal from the input unit (402);
(ii) compare the first signal with provided input parameters to determine an intestinal health of the subject;
(iii) determine a gastro-intestinal health of a subject by differentiating Congestive Colon Failure from pancreatic and colon-associated diseases; and
c. an output unit (406) coupled with the processing circuitry (404) and configured to:
(i) relay the gastro-intestinal health of the subject on an audio-visual device of the output unit (406).

12. The toilet system (400) of claim 11, wherein the input unit (402) further comprising:
(i) an imaging unit (408) configured to capture images;
(ii) a set of detectors (410) configured to sense temperature, texture, color, consistency, flow and a combination thereof, and
(iii) an audio unit (412) configured to receive sound associated with an ejection of the human waste.

13. The toilet system (100) of claim 11, wherein the input unit (402) is adapted to receive input parameters provided by the user, such that the processing circuitry (404) compares the first signal with the provided input parameters and further determines the intestinal health of the subject.

14. The toilet system (400) of claim 13, wherein the input parameters are one of, color of feed, pH of feed, ingredients of feed, or texture of feed.

15. The toilet system (400) of claim 11, wherein the processing circuitry (404) is configured to compare the input parameter representing color of feed with the set of attributes representing the color of human waste, such that the processing circuitry (404) is adapted to differentiate Congestive Colon Failure from pancreatic and colon associated diseases.

16. A clip device (500) for detecting and monitoring biological parameters of a subject, the clip device (500) comprising:
one or more binding portions (502A-502N), each binding portion of the one or more binding portions (502A-504N) is adapted to clip on a wall of a gastrointestinal track of a subject; and
a carrier portion (504) comprising:
a diode (508) that is housed in the carrier portion (504), wherein upon implanting the clip device (500) in the gastrointestinal track, the diode (508) is adapted to release from the carrier portion (504), and further adapted to detect signals representing one or more biological condition of the subject.

17. The clip device (500) of claim 16, wherein the clip device (500) is inserted into the gastrointestinal track of the subject by way of an endoscope.

18. The clip device (500) of claim 16, wherein the diode (508) is adapted to communicate with a processing circuitry by way of a transceiver.

19. A method (600) of detecting Congestive Column Failure, the method comprising:
inserting (602), by way of endoscopy insertion, a clip device (500) on a gastrointestinal track of a subject;
implanting (604), by way of one or more binding portions (502A and 502B), the clip device (500) on walls of the gastrointestinal track of the subject;
releasing (606), upon implanting, diode (508) from a carrier portion (504) of the clip device (500);
detecting (608), by way of the diode (508), signals representing one or more biological parameters associated with the gastrointestinal track of the subject;
receiving (610), by way of processing circuitry, the detected signals from the diodes (508);
comparing (612), by way of the processing circuitry, the received detected signals with prestored biological parameters; and
analyzing (614), by way of the processing circuitry, the compared data to determine a biological condition of the subject.
